(19) United States
(12) Patent Application Publication
(10) Pub. No.: US 2003/0196885 A1
(43) Pub. Date: Oct. 23, 2003

(54) Electromagnetic energy driven separation methods

(76) Inventors: Kevin S. Marchitto, Golden, CO (US); Stephen T. Flock, Arvada, CO (US)

Correspondence Address: Benjamin Aaron Adler, ADLER & ASSOCIATES, 8011 Candle Lane, Houston, TX, 77071, US

(21) Appl. No.: 10/452,441

(22) Filed: Jun. 2, 2003

Related U.S. Application Data
(63) Continuation-in-part of application No. 09/572,763, filed on May 17, 2000, now Pat. No. 6,573,491.
(60) Provisional application No. 60/134,487, filed on May 17, 1999.

(51) Int. Cl. C07C 1/00

(52) U.S. Cl. 204/157.15

(57) ABSTRACT

The present invention provides electromagnetic energy driven separation methods and molecular modification, including methods for separating molecules in a mixture, for increasing diffusion rate of a substance in a medium, for moving fluids on a substrate, for creating microvolumes of molecules, for modifying biomolecules and pharmaceuticals, and for affixing substances to a substrate. Such methods work with extremely small volumes of target and may be used for medical diagnosis and treatment.

### CROSS-REFERENCE TO RELATED APPLICATION

This application is a continuation-in-part of non-provisional application U.S. Ser. No. 09/572,763, filed May 17, 2000, which claims benefit of provisional U.S. Ser. No. 60/134,487, filed May 17, 1999, now abandoned.

### BACKGROUND OF THE INVENTION

1. Field of the Invention

The present invention relates generally to the field of electromagnetic energy driven separation methods and electromagnetic field modifications of biomolecules or pharmaceuticals. More specifically, the present invention relates to molecular separations and creation and movement of small volumes of materials, and to altering the physical and chemical properties of biomolecules with a material in proximity to the biomolecule or pharmaceutical that absorbs energy from an ambient electromagnetic field.

2. Description of the Related Art

Though a multitude of refinements have been developed to improve the distinctness of partitioning and to increase the partiton rate, better resolution of molecular species continues to be sought. This is particularly true when complex cell or protein populations, such as biological fluids, are being separated. In gel filtration terms, the elution time is decreased for these species while the zones are narrowed. In microchip processing terms, fluids are pumped along channels formed in semiconductor substrates in devices such as microchip arrays used for diagnostic testing or for high throughput screening. The channels may be created by conventional means such as chemical etching or lithography.

Although the prior art demonstrates that some of the known techniques have been used successfully in separating molecules or moving fluids along channels in semiconductor devices, a need exists in the art to develop new techniques for or to improve existing techniques for more efficient and reliable separation. Additionally, the inventors have recognized a need in the art for techniques for moving a plurality of samples into a microarray of a plurality of wells connected by one or more channels for the purpose of testing or synthesis of samples.

The prior art is deficient in the lack of effective means of separating molecules in tiny and precise volumes by applying electromagnetic energy. Furthermore, the prior art is deficient in the lack of effective means of moving an accurate volume of fluid to appropriate chambers on a microarray by applying the electromagnetic energy. Additionally, the prior art is deficient in the lack of a means to alter biomolecules or pharmaceuticals with the use of an enhancing agent and ambient electromagnetic field. The present invention fulfills this long-standing need and desire in the art.

### SUMMARY OF THE INVENTION

The present invention is directed to a method for separating molecules in a mixture. The mixture, which further comprises an enhancing agent linked to or in close proximity to the molecules in the mixture, is applied to a support whereupon the mixture is irradiated with radiofrequency energy, microwave energy, or infrared light.

The present invention also is directed to a method for increasing the diffusion rate of a substance in a medium by applying radiofrequency energy, microwave energy or infrared energy to said medium comprising the substance and further comprising an enhancing agent linked to or in close proximity to the molecules in the mixture. The radiofrequency energy, microwave energy or infrared energy generates a propagating pressure wave, a dipole force or an incoherent force in the medium, thereby increasing diffusion rate of the substance.

The present invention is directed further to a method for generating droplets from a fluid. The method comprises the steps of applying electromagnetic energy to a medium surrounding a pool of said fluid which comprises a composition of interest, generating a force on the pool of the fluid upon application of the electromagnetic energy, and moving a field generated by the force in relation to a location of said fluid in the pool. The generated force counters the surface tension of the fluid thereby releasing droplets from the pool of the fluid.

The present invention is directed further to a method for a method for altering the affinity of molecules for a substrate or separation matrix. The method comprises linking a particle to the molecules to form a molecular complex and irradiating the molecular complex with radiofrequency energy, microwave energy, infrared light, or a radiofrequency magnetic field. An interaction of the particle with the radiofrequency energy, microwave energy or infrared light alters the affinity of the molecules for the substrate or for the separation matrix.

The present invention is directed further to a method for creating small droplets containing an analyte. The method comprises linking a metallic nanoparticle to the analyte in a fluid to form a complex and irradiating the complex with radiofrequency energy, microwave energy, or infrared light. An interaction of the metallic nanoparticle in the complex with the radiofrequency energy, microwave energy or infrared light creates the small droplets containing the analyte.

The present invention is further directed to a composition having formula X-R where X is a molecular species that is to be used in a reaction and R is an enhancing agent that enhances absorption of electromagnetic energy by the composition. The composition may be used in a method to enhance the reactivity of a molecular species by applying electromagnetic energy thereto.

Other and further aspects, features, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1 shows a source of electromagnetic energy (laser pulse) impinging on an absorber, which translates the energy into a pressure wave that exerts force in the indicated directions.

FIG. 2 shows the delivery of photons that are absorbed or reflected off molecules, thereby exchanging momentum with a net force in the direction of the light.

FIG. 3 shows the formation of an optical trap and resultant force on a molecule suspended in a matrix. Movement of the trap results in an additional force that creates a pressure effect, which essentially “pulls” the molecules along a gradient.

FIG. 4 is a waveguide device for channeling energy that creates a force on particles or fluids in the waveguide.

FIG. 5 is a surface with channels showing how a molecule (or fluid) can be manipulated to move (along the dashed line) preferentially in a desired direction, by pressure devices such as optical traps.

FIG. 6 shows that pressure waves may be applied to a chemical composition in a formulation in contact with, or impregnated in, the separation media in order to drive the substances in the formulation through the medium.

### DETAILED DESCRIPTION OF THE INVENTION

In one embodiment of the present invention, there is provided a method for separating molecules in a mixture comprising the steps of applying the mixture to a support where the mixture further comprises an enhancing agent linked to or in close proximity to the molecules in the mixture and irradiating the mixture with radiofrequency energy, microwave energy, or infrared ight. The method further comprises the independent steps of optionally cooling the support and of inductively heating the enhancing agent to affix the applied molecules to the support.

In all aspects of this invention the support may be a liquid or semi-solid support. The enhancing agent may be a particle or other molecule that is different from the molecules to be separated in the mixture. Representative examples of the other molecule are water, a carbohydrate, a protein, a nucleic acid, a lipid, an amino acid, carbon dioxide, or indocyanine-green. Representative examples of particles are a ferromagnetic material, a semiconductor, silicon, tantalum, niobium, zirconium, titanium, Phynox, palladium/cobalt alloy, magnetite, nitinol, nitinol-titanium alloy, titanium alloyed with aluminum and vanadium, zirconium, aluminum oxide, cobalt, cobalt alloyed with chromium and molybdenum, cobalt alloyed with chromium, molybdenum and nickel, iron, nickel, gold, palladium, stainless steel, conductive microspheres, calcium-phosphate microspheres, magnetic microspheres, metallic coated microspheres, ruthenium, cadmium selenide, gold maleimide, hydroxysuccinimidyl gold.

Further in this embodiment, the enhancing agent increases the absorption of or the scattering of the radiofrequency energy, the microwave energy or the infrared light. Also, the radiofrequency energy, the microwave energy or the infrared light applied to the mixture creates a pressure wave, a dipole force or an incoherent force. These forces are determined by the increase of the absorption or of the scattering of the irradiative energy.

In another embodiment of the present invention, there is provided a method for increasing the diffusion rate of a substance in a medium, comprising the step of applying radiofrequency energy, microwave energy or infrared energy to the medium comprising said substance, where the medium further comprises an enhancing agent linked to or in close proximity to the molecules in the mixture such that the radiofrequency energy, the microwave energy or the infrared energy generates a propagating pressure wave, a dipole force or an incoherent force in the medium, thereby increasing diffusion rate of the substance. In all aspects of this embodiment the enhancing agents and the effect of the enhancing agents are as described supra. Furthermore, in all aspects the medium may be a semi-solid or a liquid medium.

In yet another embodiment of the present invention, there is provided a method for generating droplets from a fluid comprising the steps of applying electromagnetic energy to a medium surrounding a pool of the fluid which comprises a composition of interest; generating a force on the pool of the fluid upon application of the electromagnetic energy; moving a field generated by the force in relation to a location of the fluid in the pool wherein the generated force counters the surface tension of the fluid thereby releasing droplets from the pool of the fluid. This embodiment further comprises the independent steps of optionally cooling the medium and of moving the droplet through the medium via the field generated by the force.

In an aspect of this embodiment the fluid may comprise a pharmaceutical composition to deliver a drug contained therein. Additionally, the fluid may be a hot-melt comprising the composition of interest. Furthermore, the fluid may comprise a solvent which evaporates thereby leaving dry particles comprising the composition of interest. In all aspects of this embodiment the droplet may have a diameter of less than 100 microns.

In still another embodiment of the present invention there is provided a method for altering the affinity of molecules for a substrate or separation matrix comprising the steps of linking a particle to the molecules to form a molecular complex and irradiating the molecular complex with radiofrequency energy, microwave energy, infrared light, or a radiofrequency magnetic field; wherein an interaction of said particle with said radiofrequency energy, microwave energy, infrared light, or radiofrequency magnetic field alters the affinity of the molecules for the substrate or for the separation matrix. In all aspects of this embodiment the particle may be a strong infrared absorber, electrically conductive or magnetizable. Examples of the particle are a metallic nanocrystal, a ferromagnetic, a metal, a metal alloy, or indocyanine green.

In still another embodiment of the present invention there is provided a method for creating small droplets containing an analyte, comprising the steps of linking a metallic nanoparticle to the analyte in a fluid to form a complex and irradiating the complex with radiofrequency energy, microwave energy, or infrared light wherein an interaction of the metallic nanoparticle in the complex with the radiofrequency energy, microwave energy or infrared light creates the small droplets containing said analyte.

In still another embodiment of the present invention there is provided a composition having formula X-R, where X is a molecular species that is to be used in a reaction and R is an enhancing agent that enhances absorption of electromagnetic energy by the composition. The molecular species X may be biologically active, for example, a pharmaceutical or may be a biomolecule. The enhancing agent R may be magnetically permeable or may be sensitive to radiofrequencies. Furthermore, both X and R may be adsorbed to activated carbon.

In still another embodiment of the present invention there is provided a method of enhancing the reactivity of a molecular species comprising the steps of adding an enhancing agent to the molecular species to form the X-R composition described supra and exposing the composition to electromagnetic energy such that exposure thereto enhances the reactivity of the molecular species comprising the composition. The molecular species may be a biomolecule. The electromagnetic energy may be radiant energy, microwave energy or radiofrequency energy. Optionally, the electromagnetic energy may be applied inductively. Reactivity of the molecular species may be enhanced through migration potential or by heating.

The present invention describes methods and devices for delivering electromagnetic energy to move fluids and compounds through various separation media or for the purpose of delivering fluids in small quantities. Specifically, the invention describes methods and devices for separating compounds in various media as a result of imparting electromagnetic energy to create impulse transients, creating direct pressure on molecules based on absorption or reflection characteristics, or by creating optically active derivatives of compounds which migrate in a medium as a result of pressure imparted from a source of electromagnetic energy. Further described are microfluidic devices which utilize electromagnetic energy to create a pressure on fluids thereby allowing for manipulation of extremely small volumes.

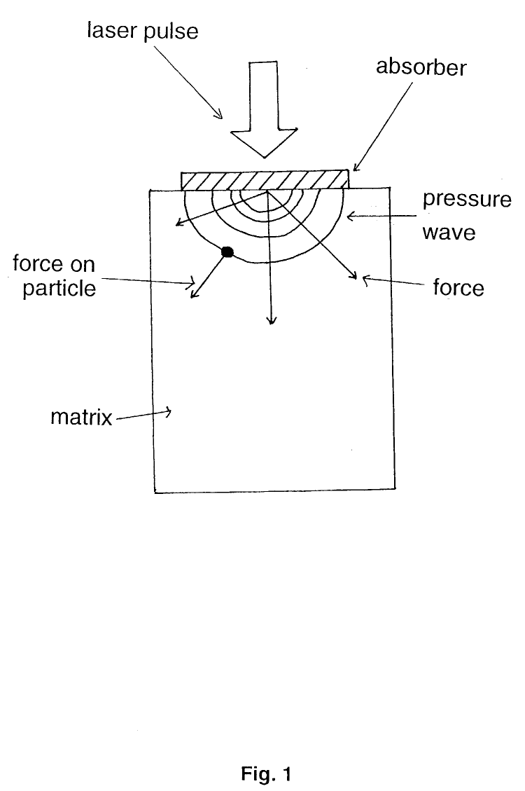

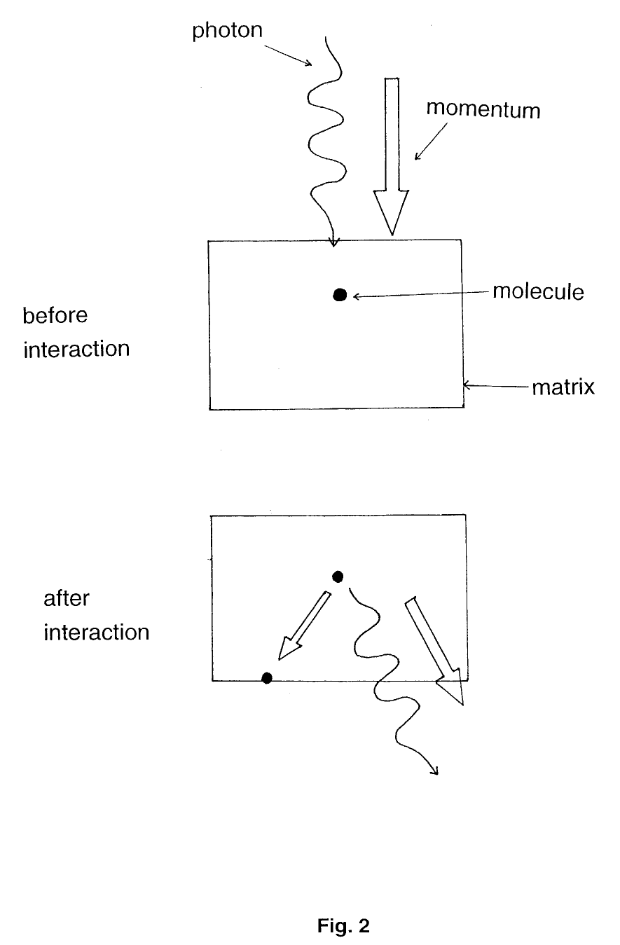

The present invention uses non-ionizing radiant energy, e.g., the infrared radiant energy produced by an Er:YAG laser, radiofrequency energy or microwave energy, to accelerate the diffusion rate of substances in semi-solid or liquid media or to accelerate the movement of droplets. These methods, referred to as delivery of optical pressure, of optical pumping or of optical propulsion, involve the creation of a type of pressure which serves to increase the pressure upon a target. When using optical pressure, propagating pressure waves are used to create pressure in a medium such that the diffusion rate of the substances in the medium, e.g. a biological compound, is increased relative to its surrounding environment (FIG. 1). In a related, but distinctly different, process using optical propulsion, the pressure associated with propagating photons can also be applied directly to compounds in order to push these substances through a semi-solid, porous or liquid medium. Optical propulsion relies on the delivery of photons that are absorbed or reflected off molecules, thereby exchanging momentum with a net force in the direction of the light (FIG. 2).

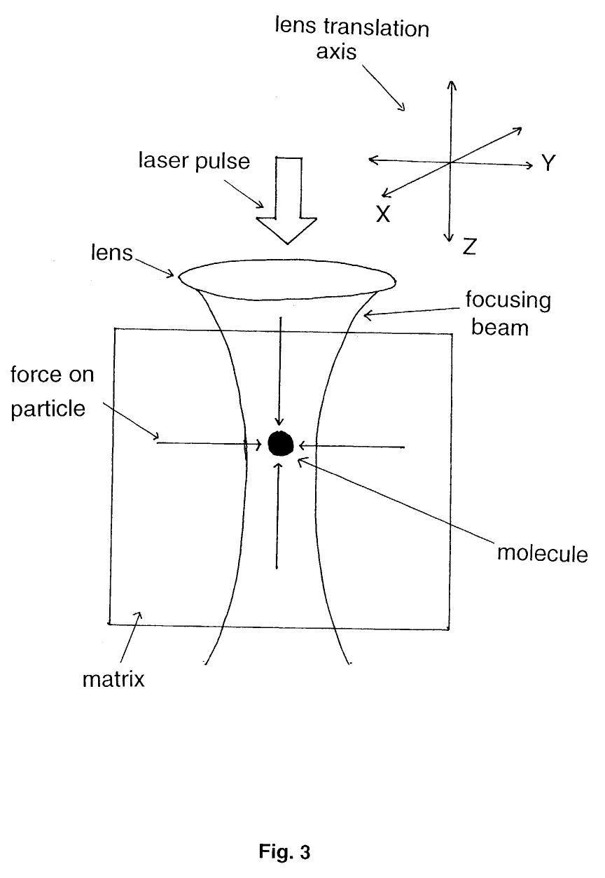

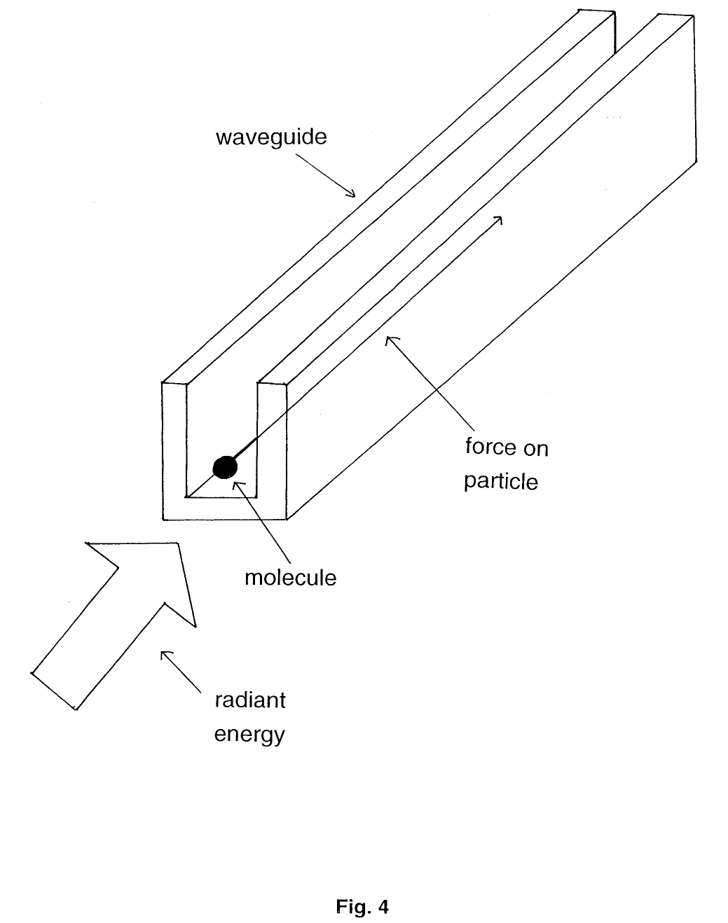

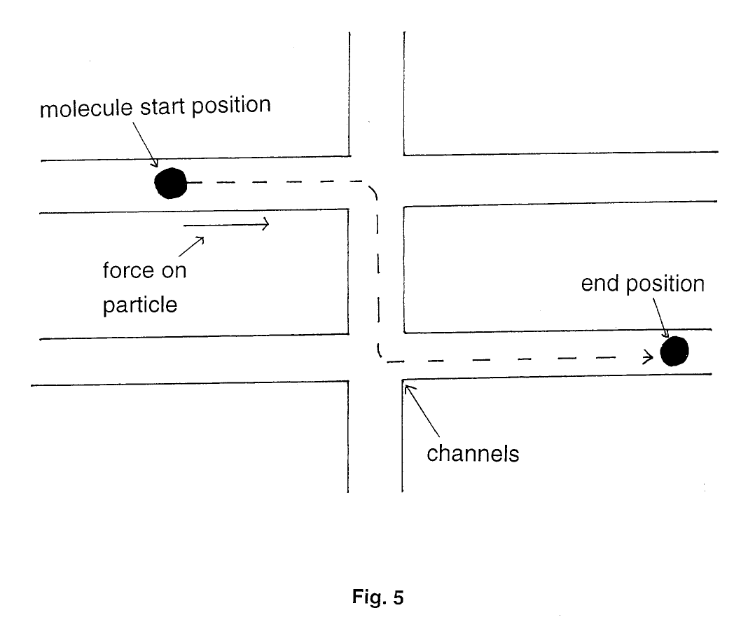

Alternatively, an optical trap is formed to create the pressure effect, which essentially “pulls” the molecules along a gradient (FIG. 3). The trapping and propulsion effects may also be used to exert pressure upon droplets, causing them to move, along a surface, in channels (FIGS. 4 and 5) or through micro-bore tubing. The radiant energy is provided by lasers, or other forms of electromagnetic radiation such as radiofrequencies or microwaves.

The present invention typically consists of a continuous wave (CW) or pulsed laser, used alone or in combination, which is capable of generating impulse transients or a focused beam of energy. In the case of impulse transients, a pressure wave is created that moves through a medium, thereby exerting a pressure that varies throughout the medium, having an effect such that particles and molecules in the medium flow at differential rates through the medium. A number of parameters may be manipulated for a desired effect on different types of solutions or chemical compositions. Some of these include energy fluence, energy fluence rate, pulse length, wavelength of radiant energy, irradiation field size, and pulse repetition rate.

An object of the present invention is to pump fluids along channels formed in semiconductor substrates in devices such as microchip arrays used for diagnostic testing or for high throughput screening. The channels may be created by conventional means such as chemical etching or lithography. These channels may be formed physically, as in the form of a trough, or alternatively, by applying a reflective material in a strip along a path the channel would take which thereby guides the electromagnetic energy.

This system is used for moving a plurality of samples into a microarray of a plurality of wells connected by one or more channels for the testing or synthesis of samples. Fluid flow is controlled by optical devices or by other types of devices that use electromagnetic energy to create a type of “pressure.” Samples may be loaded from a single or multiple loading channel and may be processed independently or in parallel. Thus, tiny, precise volumes to be manipulated such that rate of flow, volume, size of droplet and spatial delivery of the sample can all be controlled so as to deliver an accurate volume of fluid to the appropriate chambers on the array.

Droplets of varying size and volume may be formed as a consequence of the pressure generated on a fluid or material. Various parameters may be used to control droplet size, including the pressure generated directly on the material of fluid, the dipole force created in the surrounding environment, the rate at which the droplets are propelled through a medium as a result of these pressures and forces. Apertures may be designed which contribute to the formation of droplets, and which also help to control volume. Alternatively, pressure of force can be manipulated with respect to surface tension in a fluid, which in turn results in the generation of droplets of desirable size and volume.

Further provided is a means for the manufacture of particles, such as nanoparticles, of various compositions. Such compositions may be used as drug delivery agents whereby the particles contain a drug and possess characteristics that are desirable for the delivery of those drugs in a patient. These characteristics can include, for example, a desirable size that influences absorption of particles and droplets across membranes. Other controllable chracteristics include compositions that result in decreased or increased rate of dissolution in a medium.

Drug delivery compositions may include micro- or nanoparticles of a desired size used for inhalation or particles of desired size and composition that provides certain release characteristics. For example, a formulation may exist in a liquid form under one set of environmental or chemical conditions, e.g., determined by, but not limited to, chemical composition or temperature. For example, the composition may melt when exposed to heat or may be liquid when the concentration of a solvent is increased. When the environment is altered in this example, the formulation solidifies. In this manner, droplets of a desired volume may be generated while the material is in liquid form, and, upon alteration of parameters, the droplets solidify, generating particles of a desired size.

In some cases, it may be beneficial to link the molecules to be separated with another molecule or enhancer, nanocrystal or other particle in order to alter the scattering or absorbing properties of the material to be separated or moved. If linking is not an option, then mixing the enhancer and molecules so that the enhancer is in close proximity to the molecules. For example, a strong infrared absorber, such as indocyanine green, could beneficially be coupled to an antibody which has an affinity for a particular antigen on a molecule, thereby greatly increasing the absorption of the molecule and so enhancing its efficiency to be separated with infrared light. Similarly, an enhancer made up of a nanocrystal of conductive material, such as nickel, could be linked to a molecule, e.g., DNA. Subsequently a radiofrequency, electric or magnetic field would, for example, strongly affect the diffusion properties of the enhancer and molecule thus improving the ease of separating or moving the molecule. When the enhancer, e.g., metallic nanocrystal, is coupled to a protein, and the enhancer is then exposed to a radiofrequency electromagnetic field, then changes in the physical and chemical nature of the protein would result. These changes could be useful in fusing tissues or other biological materials. A similar approach involving an enhancer could be used to thermally active certain pharmaceuticals or to thermally disrupt drug delivery materials such as liposomes or microspheres.

Other enhancers may comprise water, carbon dioxide, CFCs, methane, sulphur hexafluoride, conductive nanospheres, polystyrene microspheres, or magnetic particles such as magnetite optionally coupled to poly(D,L-lactic acid). Metallic enhancers may comprise tantalum, niobium, zirconium, titanium, and platinum which are some of the most biocompatible elements. Other metal alloy enhancers may be phynox which is an alloy of cobalt, chromium, iron, nickel, molybdenum, palladium/cobalt alloy, magnetite, nitinol which is a shape memory alloy, nitinol-titanium alloy, titanium which optionally may be alloyed with aluminum (6%) and vandium (4%), aluminum oxide, cobalt which optionally may be alloyed with chromium, molybdenum and nickel or optionally 96%Co/28% Cr/6%Mo alloy, gold, palladium, and stainless steel such as biocompatible type 316L).

Additionally, the medium used in separations, or moving molecules, or the substrate on which the molecules are moved may be adjusted to exhibit properties conducive to the process for which it is being used. For example, adjusting the pH of the medium or substrate would allow for a molecule of a particular pH to have a weaker affinity for the medium. A reduction of viscosity would make samples easier to move. A reduction in surface tension would significantly alter the ability of samples to be moved around on a substrate. The substrate may optionally be cooled in order to minimize evaporation of the sample.

An enhancer substance, such as a metallic nanocrystal, can be attached to the molecules or an analyte to be separated or moved and, with the use of an external radiofrequency magnetic field, can be heated such that their affinity for the substrate or separation matrix changes. The enhancer is such that it absorbs or interacts with the applied electromagnetic field whether electric, magnetic or infrared. This affinity change can be either a reduction or an increase in affinity, optionally to the point where the analyte is bonded to the substrate or matrix. Alternatively, the nanocrystals can be replaced with metal quantum dots, metallofullerenes or metallic nanotubes.

Optionally, it may be beneficial to incorporate a cooling mechanism, such as a peltier element, into the substrate, in order to prevent evaporation of the material being moved or separated. Furthermore, the radiofrequency energy, microwave energy or infrared light, may be applied to the separation medium non-uniformly such that the material being moved or separated experiences a force dependent on spatial position. In certain situations, this may serve to enhance separation resolution analogous to gradient gel electrophoresis.

The present invention also encompasses a class of biomolecules whose reactive properties are enhanced through the addition of absorptive groups such that, upon application of electromagnetic energy, there is a resultant increase in the energy of the biomolecules. Optionally, the applied energy may be the result of inductive coupling of an electromagnetic field with the enhancers. The enhancers may be used to enhance sensitivity to electromagnetic energy resulting in augmentation of molecular vibration, heating and migration potential of a particular species. Particularly, electromagnetic energy may be used to heat a biomolecular species, with added enhancers, in solution, or in a mixture of materials, such as in tissue.

Coupling an electromagnetic field enhancer with a biomolecule can be achieved in a number of ways, for example magnetic microbeads can be coupled through a number of reactions involving, for example, strapavidin, amine, carboxyl or antibodys (Pierce Biotechnology, Rockford Ill.; Seradyne, Indianapolis, Ind.). Alternatively, activated carbon can be combined with an enhancer, e.g., gold, and a biomolecule such as a pharmaceutical, whereupon adsorption takes place resulting in a carbon-enhancer-pharmaceutical complex.

The following examples are given for the purpose of illustrating various embodiments of the invention and are not meant to limit the present invention in any fashion.

### EXAMPLE 1

Pressure Wave Optical Pressure

Pressure waves created through the interaction of electromagnetic energy with matter may be used to drive molecules through a semi-solid, porous medium or liquid such as one found in separation gels including agarose, polyacrylamide and numerous types of cellulose. For example, the interaction of laser irradiation with tissue can lead to the generation of propagating pressure waves which are generated from a rapid volumetric change in the medium by heating or by the generation of plasma. The propagating pressure waves are in the form of low-pressure acoustic waves propagating at the speed of sound or high pressure shock waves propagating at supersonic speeds. These waves also can result from generation of waves in a target that is in intimate acoustic contact with the separation media.

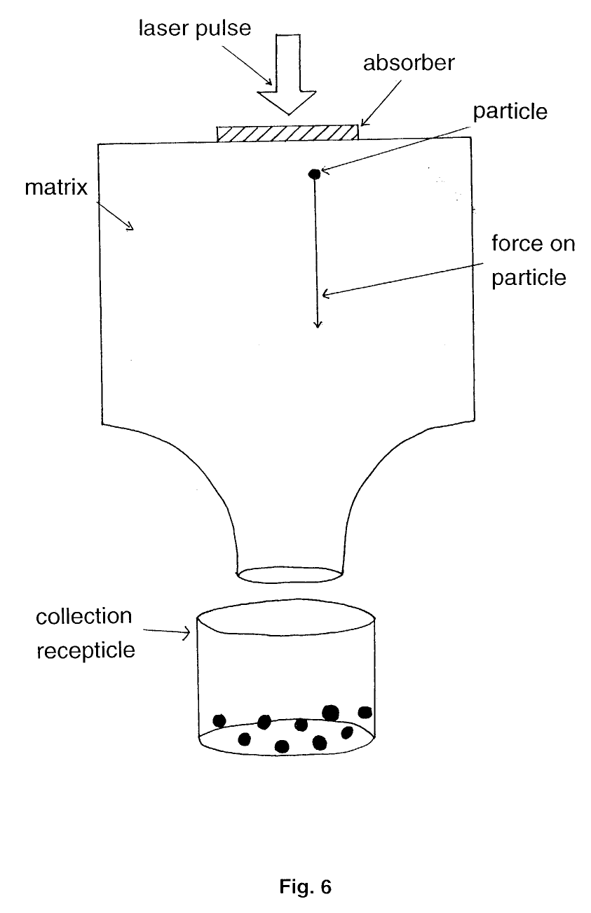

These pressure waves may be applied to a chemical composition in a formulation in contact with, or impregnated in, the separation media in order to drive the substances in the formulation through the medium (FIG. 6). Continuously pulsing electromagnetic energy delivered in discrete short duration pulses propagates the pressure waves, which thereby creates a pressure that physically forces the substances in the formulation between “pores” in the media. The pumping effect may occur through the creation of increased pressure, including osmotic or atmospheric pressure. A separation results, which is due to the differential resistance of the medium relative to the fluid medium, which is the mobile phase.

### EXAMPLE 2

Optical Propulsion

The aforementioned pumping effect may occur through the creation of increased pressure, including osmotic or static ambient pressure. Significantly, the use of optical propulsion provides a means to propel uncharged molecules. Continuous or pulsatile pressure may be applied directly to particles and molecules themselves in a medium.

The particulate or molecular target has absorption or scatter characteristics different than those of the medium. The absorption or scatter of electromagnetic energy of the target results in an exchange of momentum from the photons to the target such that the target is propelled at a differential rate relative to the medium. Optimally, the wavelength chosen would neither result in a molecular nor electronic rearrangement as these two events would lead to the inefficient use of energy. Light can exert forces on a molecule because photons carry momentum. The exchange of photon momentum with a molecule can occur incoherently, as in the absorption and readmission of photons, or coherently, as in the redistribution of or lensing of the incident field by the molecule.

### EXAMPLE 3

Incoherent Force

The incoherent interaction that can alter the momentum of an atom is also called the scattering force because it arises from direct scattering events. Every time an atom scatters a photon carrying momentum p=h/λ where h is Planck's constant and λ is the wavelength of light, the atom experiences a small change in velocity. In the case of incoherent scattering two momentum impulses are delivered to the atom with one along the direction of the incident photon and another opposite the direction of the scattered photon. Because the photons in rare media are not scattered into a preferred direction, the net average velocity change per scattered photon Δv is opposite the direction of the incident photons with λv=p/M=h/λM, where M is the mass of the atom. Note that this force therefore also provides a means with which to separate atomic or molecular species based on their mass, M.

The momentum imparted on the molecular target in an inelastic collision is equal to the ratio of the photon energy, U, divided by the speed of light, c. Given a critical amount of energy fluence, i.e., rate, in the electromagnetic energy continuous-wave beam or pulse, significant forces can be imparted on the molecular target thereby inducing movement since force is equal to the time derivative of momentum.

This incoherent force may be used to enhance passive diffusion. The electromagnetic energy, produced by a pulsed or continuous-wave Nd:YAG laser at 1064 nm wavelength, irradiates a molecule which does not significantly absorb energy having such wavelength. The molecule, if placed on the a semi-solid support such as gelatin or agarose, scatters the electromagnetic energy in such a way that the net momentum imparted upon the molecule is in a direction away from the surface of the support. Thus, the penetration of the drug into material would be enhanced as compared to passive diffusion.

The incoherent force on a molecule results when the molecule absorbs or scatters radiant energy. Such force is the momentum associated with photons and not a pressure wave pushing the molecules. Incoherent force is the most efficient when absorption takes place, although it is important that the molecule doing the absorbing does not undergo an irreversible change such as photolysis or chemical bond-breakage.

### EXAMPLE 4

Coherent Force

The force arising from a coherent interaction with light is also called the dipole force. The laser field polarizes the atom which then experiences a force in the gradient of an electromagnetic field. The strong electric field of a laser beam can be used to induce a dipole moment in a process called optical trapping. As long as the frequency of the laser field is below the natural resonances of the particle being trapped, e.g. below the atomic transition of an atom or the absorption band of a polystyrene sphere, the dipole moment is in phase with the driving electrical field. Because the energy, W, of the induced dipole, p, in the laser field, E, is given by W=−pE, the particle achieves a lower energy state by moving into the high-intensity focal spot of the laser beam.

There have been numerous reports of optical traps being used to manipulate particles or even cells. These traps are used to move these tiny particles around under a microscope objective. Optical tweezers have also been described whereby a focal spot of a single beam optical trap is moved with mirrors or lenses. It has also been shown that other forms of electromagnetic energy may be used to form such traps.

As used herein, a trap is made by creating a non-uniform field of waves, e.g. photons, ultrasound, electric or magnetic. Due to the interaction of the molecule with the non-uniform field, there is a force imparted that tends to pull the molecule towards the most intense part of the field. Such a trap is formed at the tissue interface where a desired molecular target is to be moved in a particular direction.

In the case of separations, the direction is into a semi-solid support. Thus, the focal point of the trap is moved along a vector that penetrates the material of interest, while a solution containing the compounds to be separated is applied to the surface of the tissue. In the case of an optical trap, the focal point of a single beam or multiple beam trap would then be moved progressively into the matrix of the support, which could occur cyclically so as to ensure the maximum pumping effect. The most intense part of the field, typically the focal point of some optic, is moved in a way such that the molecules are dragged into or out of the tissue. This method allows for continuous and controllable transmembrane drug delivery.

Besides optical traps, other types of traps, such as magnetic, radiofrequency or microwave traps would also be useful. Radiofrequency or microwave radiant energy would be most suitable as the physical size of the volume whereby a driving force could be created is much larger than it is when light is used. Optical traps using light are microns in size, while traps using microwaves or RF could be centimeters in size. Furthermore, because microwaves and RF are not scattered in tissue as much as light, the former would be able to maintain their integrity to a greater depth in tissue than light.

### EXAMPLE 5

Target Molecules/Compounds

Strategies described herein include targeting molecules or molecular groups based on energy absorption characteristics. Absorption typically depends on the functional group present and not the complete molecular structure or absorption may be due to single bond stretching and bending vibrations. For example, infrared absorption of biomolecules can be broadly broken down into three regions. An OH group stretching vibration is near 7140 cm−1 (1.4 microns) and an NH stretching vibration is near 6667 cm−1 (1.5 microns) in the near-infrared or NIR (800 nm-1.5 microns). In the mid-IR, 4000 to 1300 cm−1 (2.5-7.7 microns) is found the group frequency region, while 1300 to 650 cm−1 (7.7-15.4 microns) is the fingerprint region. Choosing groups that optimally absorb or reflect radiation allows the motion characteristics of the molecule carrying that group to be optimized.

Also, adding absorbing groups to molecules may enhance their sensitivity and migration potential. Specific formulations are chosen such that electromagnetic energy absorption or scatter is maximized relative to the surrounding medium. Further, many compounds may be modified by the addition of such energy absorbing or scattering groups so as to maximize optical propulsion of a particular formulation.

Therefore a new class of compounds that are optically propelled by virtue of the addition of groups or structures that absorb or scatter light in a characteristic way that imparts momentum to the molecule causing it to move relative to the medium which contains it are defined. These compounds are designed to include both physiologically active groups and molecular groups which maximize the absorbance or scatter of light so as to be propelled relative to its surrounding medium. For example, it is possible to conjugate a photosensitizing compound, e.g., CMA, with an antibody in the region of the antibody whereby the ability of the antibody to bind to the antigen is not inhibited. CMA strongly absorbs radiant energy in the visible region of the electromagnetic spectrum, e.g. 650 nm, and so can be used in conjunction with incident 650 nm radiant energy to propel the antibody.

Any alteration in a molecule, such as dimerization or the addition of a group, will change the absorption and scattering properties of the molecule. An increase in either will increase the efficiency of the dipole trap. For example, addition of magnetic species, such as ferro-, para- and diamagnetic, will enhance the effect of magnetic fields on the molecule. Alternatively, acoustical properties of molecules can be changed by addition of contrast enhancers such as galactose. The addition of this molecule would enhance the magnitude of the push. When using the coherent force to move molecules, it may be beneficial to alter the molecule by enhancing it's scattering cross section through the conjugation of a molecule to, for example, decrease the wavelength of resonance or decrease the natural lifetime.

Specific compound formulations are selected so that electromagnetic energy absorption is maximized relative to the surrounding medium. Many pharmaceutical or diagnostic compounds can be modified by the addition of energy absorbing groups to maximize the effects of the electromagnetic energy on a particular formulation relative to the surrounding medium. A new class of compounds may be defined by their unique permeability and migration characteristics in the presence of or following a treatment of electromagnetic energy. These molecules possess different characteristics by virtue of the addition of energy absorbing structures. As a result, the molecules are imparted momentum to move relative to the surrounding medium or are altered due to the excitation of the molecules. Rapid heating of a molecule preferentially absorbing energy relative to its environment by radiofrequency or microwave energy may result in cleavage of a heat-sensitive linkage or activation of a specific activity. These compounds are designed to activate molecular groups that maximize the absorbance or reflectance of energy to achieve the desired effect.

Similarly pharmaceutically active compounds may be modified by the addition of groups that readily form a dipole when exposed to appropriate electromagnetic energy, such as laser light, radiofrequencies or microwaves. The addition of such groups thus would result in enhanced ability to use optical trapping methods for the delivery of these types of compounds as described herein.

For example, some dielectrics, such as polystyrene, can be induced to form dipoles in the presence of an electromagnetic field and, when in the form of polystyrene microbeads, can also be conjugated to proteins when they are coated with molecules with an affinity to proteins. An example is avidin-coated polystyrene beads which efficiently conjugate to biotinylated linked protein. Thus, any compound which may interact with electromagnetic energy in such a way that it is propelled through a medium may be used. This provides a means by which molecules may be propelled through a medium at differential rates relative to the medium and other molecules in the medium and a means by which molecules may be separated from one another based on their characteristic interaction with electromagnetic energy.

### EXAMPLE 6

Microfluidics

Liquid droplets may also be manipulated. For example, impulse transients may be applied to a fluid on a surface or in a tube. The pressure wave created by these impulse transients works on the fluid to move it in a desired direction, generally in the direction of the energy impulse.

Alternatively, a trap may be formed that exerts a pressure at the fluid interface. Trap formation and manipulation of cells has been described. Similar traps can be used to exert pressure at the fluid interface in the same manner that Optical Tweezers trap particles in the waist of a strongly focused continuous wave laser beam. The optical trap results because the objects that are trapped in the focus of the laser beam experience a restoring force if they try to leave the high intensity volume.

The incoming beam is separated into two beams. Each beam can be characterized as a plane wave that follows the laws of optics. Most or all of the light is transmitted at the surface of the spherical droplet. If the droplet is placed below the center of the focus, the resulting force of the trap will act in the upward direction. If the droplet is placed above the center of the focus, the resulting force of the trap will act in the downward direction. If the droplet is placed to the right of the focus, the resulting force of the trap will force the object move towards the center of the trap.

A “channel” is created using two beams which impinge on the fluid from two angles, e.g., 45° and 135°. These lasers create pressure transients which essentially contain a volume, in effect providing a means for adjusting or controlling channel size. The trap then can be moved along a vector such that fluid motion is achieved. Volume displacement may be controlled by increasing or decreasing the intensity of the energy delivered.

An optical trap is formed at the surface of a solid support, such as a silicon wafer, where a desired molecular target is to be moved in a particular direction. In this case, a microfluid channel is created by directing the beam along a vector in the direction of interest. Fluids may be directed along channels which are embedded in the support or along the surface of a flat surface whereby a channel is created by the directional vector of the trap. Thus, the focal point of the trap is moved along a vector that channels the fluid in the direction of interest.

Solutions may be brought in contact with one another to form reactions, as in the case of high throughput screening procedures for drug discovery. Alternatively, mixtures of reagents may be moved along the surface of a support in microassasy preparations. The focal point of a single beam or multiple beam trap would then be moved progressively along the desired channel of the support, which could occur cyclically so as to ensure the maximum pumping effect.

### EXAMPLE 7

Pressure Waves for Driving Compounds through Semi-Solid Supports

Pressure waves created through the interaction of electromagnetic energy with tissue or non-biological matter may be used to drive molecules in a medium across semi-solid interfaces. The interaction of radiofrequency (RF), microwave irradiation or radiant energy with an absorber can lead to the generation of propagating pressure waves, which are generated from a rapid volumetric change in the medium by heating or by the generation of plasma. Propagating pressure waves are in the form of low pressure acoustic waves propagating at the speed of sound or high pressure shock waves propagating at supersonic speeds. These waves can also be a consequence of a generation of waves in a non-biological target which is in intimate acoustic contact with the biological media.

Continuously pulsing electromagnetic energy delivered in discrete short duration pulses propagates the pressure waves which thereby physically move the molecules between spaces in the matrix. The pumping effect may occur through the creation of increased pressure, including osmotic or atmospheric pressure. A separation results due to the differential resistance of the matrix relative to the fluid medium. The degree of pumping is related to the shape, duty cycle and power of the driving energy source.

The interaction of laser electromagnetic energy with a semi-solid or liquid medium can lead to the generation of propagating pressure waves in the form of low pressure waves propagating at the speed of sound or high pressure shock waves propagating at supersonic speeds. For low pressure waves, an example of efficacious irradiation parameters would be a wavelength of 1064 nm, 20 ns pulses with energy of 20 mJ and a spot size of 1 mm. For high pressure shock waves a wavelength of 2.94 microns is used with all other parameter remaining the same.

Pumping may sometimes be inefficient if the energy is deposited directly on a tissue having a large surface area. To compensate for this inefficiency, a target which preferentially absorbs energy at these frequencies may be placed adjacent to the tissue. In the case of high frequencies, this target could effectively act as an antenna and optionally may be composed of metals or metal containing compounds.

For example, a Q-switched Nd:YAG laser producing radiant energy at 1064 nm is configured to produce 20 ns pulses with energies of 2 to 20 mJ at a pulse repetition rate of 10 to 1000 Hz. The beam is focused to a circular spot with a diameter of 1 mm. A thin piece of black anodized aluminum is placed on the surface of a polyacrylamide gel and a thin layer of aqueous trypan blue (4%) or other dye positioned between the aluminum and gel surface.

The laser is oriented to irradiate the surface of the aluminum support or silicon wafer at an angle of about 15 degrees to about 45 degrees. The laser is mounted on a two-dimensional platform with micrometer controlled movement. Irradiaton and concurrent movement allow the droplet, or micro-droplet partitioned from the droplet, to migrate along a vector that is created by directional movement of the laser.

The irradiation takes place for a period of one or more hours, after which time the aluminum is removed and the trypan blue remaining on the surface was removed with an absorptive towel. The gel is cut in cross section through the center of the two positions where the trypan blue is positioned. Upon transillumination with visible light, the trypan blue is shown to have diffused further into the gel below the irradation site than where no laser irradiation took place. A related experiment where the laser is oriented so the focusing lens is at the surface of the trypan blue may be used to demonstrate the result of electromagnetic energy induced pressure resulting from molecular absorption or scatter.

### EXAMPLE 8

An Example of Optical Propulsion

Continuous or pulsatile pressure may be applied directly to particles and molecules themselves in a medium using electromagnetic energy. For example, a protein could be conjugated to readily available colored polystyrene microspheres which absorb the electromagnetic energy from a diode-pumped Nd:YAG micro-laser at 1064 nm with 1 ns pulses at a repetition rate of 10 kHz and peak power of 25 kW. Momentum is imparted unto the polystyrene sphere by the electromagnetic energy, and so it is pushed in the direction of the propagation of the electromagnetic energy. By adjusting the focal point along this directional vector, i.e., into the medium, pressure can be exerted on the particles such that they migrate through the medium. This principal can be applied to the separation of a mixture of compounds whereby different molecules migrate to a lesser or greater degree based on their unique absorption characteristics.

The optical pumping effect may occur through the creation of increased pressure, including osmotic or atmospheric pressure. To push particles or molecules in a medium, the chosen wavelength of radiant energy is not absorbed by the ambient medium. The particulate or molecular object to be propelled has different absorption characteristics than the medium such that radiant energy is absorbed on the surface of the object or internally. Optimally, the wavelength chosen result in neither a quantum mechanical molecular nor electronic rearrangement as these two events would lead to the inefficient use of energy. The momentum imparted on the molecular target is equal to the ratio of the photon energy divided by the speed of light. Given a critical amount of energy fluence, i.e., rate, in the radiant energy continuous wave beam or pulse, significant forces may be imparted on the molecular target thereby inducing movement.

### EXAMPLE 9

Optical Traps

One system utilizing an optical trap consists of a versatile inverted microscope with a set of suitable and highly magnifying objectives and with accurate and smoothly running motorized x-, y- and z-adjustment of the table, a pulsed Nd:YAG laser which produces UV light at 355 nm for similar activities, various optical components, e.g., mirrors, lenses, polarizers, polarizing splitting and merging cubes, filters, that create, inter alia, two fully movable, i.e., in the x-, y- and z-directions, optical traps within the field of view in the microscope, a system of high precision stepmotors for accurate mirror and lens movements for remote manipulation of the two optical traps, CCD cameras where one has enhanced sensitivity for studies of light sensitive processes, such as but not limited to, weak fluorescence, high resolution videos for registration and storage, computers that can handle the information provided by the CCD cameras, keep track of the positions of all stepper motors for control and visualization of the positions of the optical traps and store and handle all data and tasks.

These microfluidic methods may be applied to fluid delivery in microarray devices that are constructed upon chips with or without channels. In the absence of channels, a channeling effect may be achieved by creating pressure at the droplet interface between two beams as described above. Volume adjustments and flow parameters may be adjusted vectorally by moving beams to produce a funneling effect or by moving a trap such that flow results. In this manner, a controlled volume of fluid may be delivered to a particular site on the chip.

### EXAMPLE 10

Optical Traps to Generate Microdroplets

An optical trap is formed in the vicinity of a series of fluids with varied compositions that produce unique surface tensions with respect to one-another. A negative force associated with the generation of a dipole moment in the vicinity of the pool of fluid will counter the force generated by the surface tension of the fluid. When the dipole force exceeds the structural force imparted by the surface tension, a micro-droplet will be released from the pool.

One skilled in the art will readily appreciate that the present invention is well adapted to carry out the objects and obtain the ends and advantages mentioned, as well as those inherent therein. The present examples along with the methods and procedures described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. A method for separating molecules in a mixture, comprising the steps of:
applying said mixture to a support, said mixture further comprising an enhancing agent linked to or in close proximity to the molecules in said mixture; and
irradiating said mixture with radiofrequency energy, microwave energy, or infrared light.

2. The method of claim 1, further comprising the step of cooling the support.

3. The method of claim 1, further comprising the step of inductively heating said enhancing agent thereby affixing said molecules to the support.

4. The method of claim 1, wherein said enhancing agent is a particle or other molecule different from the molecules to be separated comprising said mixture.

5. The method of claim 4, wherein said other molecule is water, a carbohydrate, a protein, a nucleic acid, a lipid, an amino acid, carbon dioxide, or indocyanine-green.

6. The method of claim 4, wherein said particle is a ferromagnetic material, a semiconductor, silicon, tantalum, niobium, zirconium, titanium, Phynox, palladium/cobalt alloy, magnetite, nitinol, nitinol-titanium alloy, titanium alloyed with aluminum and vanadium, zirconium, aluminum oxide, cobalt, cobalt alloyed with chromium and molybdenum, cobalt alloyed with chromium, molybdenum and nickel, iron, nickel, gold, palladium, stainless steel, conductive microspheres, calcium-phosphate microspheres, magnetic microspheres, metallic coated microspheres, ruthenium, cadmium selenide, gold maleimide, or hydroxysuccinimidyl gold.

7. The method of claim 1, wherein said support is a liquid or semi-solid support.

8. The method of claim 1, wherein said enhancing agent increases the absorption of or the scattering of said radiofrequency energy, said microwave energy or said infrared light.

9. The method of claim 1, wherein said radiofrequency energy, said microwave energy or said infrared light creates a pressure wave, a dipole force or an incoherent force.

10. A method for increasing the diffusion rate of a substance in a medium, comprising the step of:
applying radiofrequency energy, microwave energy or infrared energy to said medium comprising said substance, said medium further comprising an enhancing agent linked to or in close proximity to the molecules in said mixture; wherein said radiofrequency energy, microwave energy or infrared energy generates a propagating pressure wave, a dipole force or an incoherent force in said medium, thereby increasing diffusion rate of said substance.

11. The method of claim 10, wherein said enhancing agent is a particle or other molecule different from the molecules comprising said mixture.

12. The method of claim 11, wherein said other molecule is water, a carbohydrate, a protein, a nucleic acid, a lipid, an amino acid, carbon dioxide, or indocyanine-green.

13. The method of claim 11, wherein said particle is a ferromagnetic material, a semiconductor, silicon, tantalum, niobium, zirconium, titanium, Phynox, palladium/cobalt alloy, magnetite, nitinol, nitinol-titanium alloy, titanium alloyed with aluminum and vanadium, zirconium, aluminum oxide, cobalt, cobalt alloyed with chromium and molybdenum, cobalt alloyed with chromium, molybdenum and nickel, iron, nickel, gold, palladium, stainless steel, conductive microspheres, calcium-phosphate microspheres, magnetic microspheres, metallic coated microspheres, ruthenium, cadmium selenide, gold maleimide, or hydroxysuccinimidyl gold.

14. The method of claim 11, wherein said enhancing agent increases the absorption of or scattering of radiofrequency energy, microwave energy, or infrared light.

15. The method of claim 11, wherein said medium is a liquid or semi-solid medium.

16. A method of generating droplets from a fluid, comprising the steps of:
applying electromagnetic energy to a medium surrounding a pool of said fluid, said fluid comprising a composition of interest;
generating a force on the pool of said fluid upon application of the electromagnetic energy;
moving a field generated by the force in relation to a location of said fluid in the pool;
wherein the generated force counters the surface tension of said fluid thereby releasing droplets from the pool of said fluid.

17. The method of claim 16, further comprising the step of cooling said medium.

18. The method of claim 16, further comprising the step of moving said droplet through said medium via the field generated by said force.

19. The method of claim 16, wherein the fluid comprises a pharmaceutical composition to deliver a drug contained therein.

20. The method of claim 16, wherein said fluid is a hot-melt comprising the composition of interest.

21. The method of claim 16, wherein the fluid further comprises a solvent, said solvent evaporating thereby leaving dry particles comprising the composition of interest.

22. The method of claim 15, wherein the droplets have a diameter less than about 100 microns.

23. A method for altering the affinity of molecules for a substrate or separation matrix comprising the steps of:
linking a particle to said molecules to form a molecular complex; and
irradiating said molecular complex with radiofrequency energy, microwave energy, infrared light, or a radiofrequency magnetic field;
wherein an interaction of said particle with said radiofrequency energy, microwave energy, infrared light, or radiofrequency magnetic field alters the affinity of said molecules for the substrate or for the separation matrix.

24. The method of claim 23, wherein said particle is a strong infrared absorber, electrically conductive or magnetizable.

25. The particle of claim 23, wherein said particle is a metallic nanocrystal, ferromagnetic, a metal, a metal alloy, or indocyanine green.

26. A method for creating small droplets containing an analyte, comprising the steps of:
linking a metallic nanoparticle to said analyte in a fluid to form a complex; and
irradiating the complex with radiofrequency energy, microwave energy, or infrared light; wherein an interaction of said metallic nanoparticle in the complex with said radiofrequency energy, microwave energy or infrared light creates the small droplets containing said analyte.

27. A composition having formula X-R, wherein X is a molecular species that is to be used in a reaction and R is an enhancing agent that enhances absorption of electromagnetic energy by said composition.

28. The composition of claim 27, wherein X is a biologically active molecule.

29. The composition of claim 28, wherein said biologically active molecule is a pharmaceutical.

30. The composition of claim 27, wherein X is a biomolecule.

31. The composition of claim 27, wherein R is an optical absorber.

32. The compostion of claim 27, wherein R is magnetically permeable.

33. The composition of claim 27, wherein R is sensitive to radiofrequencies.

34. The composition of 27, wherein R and X are adsorbed to activated carbon.

35. A method of enhancing the reactivity of a molecular species comprising the steps of:
adding an enhancing agent to said molecular species to form the composition of claim 27; and
exposing said composition to electromagnetic energy; wherein exposure thereto enhances the reactivity of said molecular species comprising the composition.

36. The method of claim 35, wherein reactivity is a migration potential of said molecular species.

37. The method of claim 35, wherein the reactivity is from heating said molecular species.

38. The method of claim 35, wherein said molecular species is a biomolecule.

39. The method of claim 35, wherein the electromagnetic energy is radiant energy, microwave energy or radiofrequency energy.

40. The method of claim 35, wherein the electromagnetic energy is applied inductively.
